(19) United States
(12) Patent Application Publication
(10) Pub. No.: US 2024/0252819 A1
(43) Pub. Date: Aug. 1, 2024

(54) SYSTEMS AND METHODS FOR OPTIMIZING USE OF A MEDICAL DEVICE FOR PAIN MANAGEMENT

(71) Applicant: BioWave Corporation, Norwalk, CT (US)

(72) Inventors: Bradford SIFF, Norwalk, CT (US); John CARTER, Belle Harbor, NY (US)

(73) Assignee: BioWave Corporation, Norwalk, CT (US)

(21) Appl. No.: 18/422,869

(22) Filed: Jan. 25, 2024

Related U.S. Application Data
(60) Provisional application No. 63/441,231, filed on Jan. 26, 2023.

(51) Int. Cl. A61N 1/36; A61N 1/02; G16H 40/67

(52) U.S. Cl. CPC A61N 1/36021; A61N 1/025; A61N 1/36031; A61N 1/36034; G16H 40/67

(57) ABSTRACT

A method for managing pain of a patient includes operating, by a patient computing device, an electrotherapy device communicatively coupled to the patient computing device through a patient facing application. The method may further include collecting a plurality of patient information passively from the electrotherapy device and the patient facing application, and actively from a user input. The method may further include transmitting the plurality of patient information from the patient facing application to a patient information database for analysis. The method may further include receiving a therapy recommendation from the patient information database, wherein the therapy recommendation comprises at least one new therapy session parameter. The method may further include displaying the therapy recommendation to a user of the patient computing device through the patient facing application on the patient computing device. Systems and computer readable medium with stored instructions to manage pain of a patient are also disclosed.

### FIELD OF DISCLOSURE

The present disclosure relates to optimizing the use of a medical device, such as an electrotherapy device, through the use of a pain management system configured to improve patient outcomes through patient therapy recommendations and compliancy notifications.

### BACKGROUND

Traditionally, electrotherapy devices have generated alternating current frequencies using a variety of different methods. For example, Matthews' U.S. Pat. No. 5,269,304 issued on Dec. 14, 1993 discloses an electrotherapy apparatus that includes at least two electrodes adapted to feed oscillating current to selected sites on or beneath the epidermal or mucous surface remote from the treatment site. The Matthews' Patent uses a common return electrode provided at the treatment site that is subjected to the sum of the currents from the two feed electrodes. The feed electrodes may be contact feed electrodes or capacitive feed electrodes. The feed electrodes may operate at different frequencies so that the treatment site is stimulated by the beat frequency. This may be about 80 or 130 Hz, if an anaesthetizing effect is required.

Traditional devices, such as those described in the above Matthews' Patent, have undesirable effects and deficiencies that the present disclosure solves. Another electrotherapy device, Carter, et al. U.S. Pat. No. 6,584,358 issued on Jun. 24, 2003 discloses an electro-therapy apparatus and method for providing therapeutic electric current to a treatment site of a patient, having means for providing two oscillating or pulsing electric alternating currents, of frequencies which differ from each other by as little as 1 Hz and up to about 250 Hz, but each being of frequency at least about 1 KHz. The apparatus and method require only one feed electrode adapted to feed the electric currents to selected feed sites on or beneath the epidermal or mucous surface of the patient, and only one return electrode adapted to be positioned on or beneath the epidermal or mucous surface of the patient, locally to the treatment site. This device, described in the Carter, et al. patent is undesirable because of the heat generated that results from the use of a class A/B amplifier, the large heat sink requirement, the need for ventilation holes and as a result, the inability to meet quality standards for home healthcare medical devices.

Furthermore, physicians, therapists, and/or specialists (clinicians) using a conventional therapy device for a patient's pain management therapy plan currently rely on delayed information to update how and where the patient uses the device. For example, some therapy devices may have a smartphone application that compiles passive therapy device treatment information, but that passive information is used weeks or months later (if at all) to inform a physician, therapist, specialist, or patient of how the patient has been using the therapy device. That information, once retrieved, can be used by the physician, therapist, and/or specialist to recommend an update to the patient's therapy plan. However, this recommendation is delayed at best and may allow the patient to incorrectly use the therapy device for weeks, months, or longer without correction.

Traditional electrotherapy devices are frequently used as an alternative to surgery, drugs, or other treatment methods based on the lower cost of treatment. However, greater adoption of electrotherapy devices may be seen if insurance companies, hospitals, and/or healthcare systems were given an insight to how the electrotherapy devices were used and the reported outcomes from using the electrotherapy device.

### SUMMARY

In accordance with some embodiments, a pain management system includes an electrotherapy device, wherein the electrotherapy device is configured to generate a first signal and a second signal. Each signal may have a base frequency value between 100 Hz and 500 kHz and are amplified by a respective amplifier. The electrotherapy device may also be configured to minimize the direct current (DC) component of the first signal and the second signal using a balanced amplifier. The electrotherapy device may also be configured to form a therapeutic signal configured to reduce pain at a treatment site by simultaneously sending the first signal from a first electrode to a second electrode and sending the second signal from the second electrode to the first electrode, and then simultaneously sending the first signal from the second electrode back to the first electrode and the second signal from the first electrode back to the second electrode. The first signal and the second signal may be linearly independent off phase alternating current signals. The electrotherapy device may also be configured to adjust the therapeutic signal utilizing a feedback system based on impedance changes within a body of a patient, wherein the impedance is measured across the first electrode and the second electrode. The pain management system may also include a patient computing device communicatively coupled to the electrotherapy device through a patient facing application, wherein the patient facing application is configured to operate the electrotherapy device, display directions, collect patient information, and display therapy recommendations.

The pain management system may also include a clinician computing device communicatively coupled to the patient computing device comprising a clinician facing application configured to receive transmitted patient information. The pain management system may also include a patient information database communicatively coupled to the patient computing device and the clinician computing device through the patient facing application and clinician facing application. The patient information database may be configured to receive transmitted patient information. The patient information database may be configured to store and analyze the transmitted patient information, and provides at least one therapy recommendation to a user of the patient computing device through the patient facing application based on the analysis of the transmitted patient information.

In accordance with some embodiments, a method for managing pain of a patient includes operating, by a patient computing device, a electrotherapy device communicatively coupled to the patient computing device through a patient facing application. The method may further include collecting a plurality of patient information passively from the electrotherapy device and the patient facing application, and actively from a user input. The method may further include transmitting the plurality of patient information from the patient facing application to a patient information database for analysis. The method may further include receiving a therapy recommendation from the patient information database, wherein the therapy recommendation comprises at least one new therapy session parameter. The method may further include displaying the therapy recommendation to a user of the patient computing device through the patient facing application on the patient computing device.

### BRIEF DESCRIPTION OF THE DRAWINGS

The features and advantages of the present disclosure will be more fully disclosed in, or rendered obvious by, the following detailed descriptions of example embodiments. The detailed descriptions of the example embodiments are to be considered together with the accompanying drawings wherein like numbers refer to like parts and further wherein:

FIG. 1 illustrates the hyperpolarization mechanism of pain reduction in accordance with some embodiments;

FIG. 2 illustrates the gate control mechanism of pain reduction in accordance with some embodiments;

FIG. 3 illustrates output portions of an electrotherapy device in accordance with some embodiments;

FIG. 4 illustrates the coupling of Sine wave 1 and Sine wave 2 to the electrodes when the apparatus is constructed around ground reference (local Apparatus ground) linear power amplifiers in accordance with some embodiments;

FIG. 5 illustrates the structure of an electrotherapy device in accordance with some embodiments;

FIG. 6 illustrates the general block structure of an electrotherapy device in accordance with some embodiments;

FIG. 7 illustrates the general block structure of an electrotherapy device in accordance with some embodiments;

FIG. 8 illustrates the general block structure of an electrotherapy device in accordance with some embodiments;

FIG. 9 illustrates two different exemplary electrotherapy devices 402 in accordance with some embodiments;

FIG. 10 illustrates one example of a pain management system in accordance with some embodiments;

FIG. 11 is an example computing device of a pain management system in accordance with some embodiments;

FIG. 12 illustrates wireless communication between an electrotherapy device and a patient computing device in accordance with some embodiments;

FIG. 13 is a view of a patient facing application in accordance with some embodiments;

FIG. 14 is a view of a patient facing application displaying a plurality of therapy sessions in accordance with some embodiments;

FIG. 15 is a first view of a patient facing application operating an electrotherapy device in accordance with some embodiments;

FIG. 16 is a second view of a patient facing application operating an electrotherapy device in accordance with some embodiments;

FIG. 17 is a third view of a patient facing application operating an electrotherapy device in accordance with some embodiments;

FIG. 18 is a view of a configurable therapy session displayed on a patient facing application in accordance with some embodiments;

FIG. 19 is a view of one of the plurality of usage visuals displayed on a patient facing application in accordance with some embodiments;

FIG. 20 is a first notification displayed on a patient computing device in accordance with some embodiments;

FIG. 21 is a second notification displayed on a patient computing device in accordance with some embodiments;

FIG. 22 is a view of a home dashboard on a clinician facing application in accordance with some embodiments;

FIG. 23 is a view of a therapy plan compliancy screen on a clinician facing application in accordance with some embodiments;

FIG. 24 is a therapy adherence report screen on a clinician facing application in accordance with some embodiments;

FIG. 25 is a view a screen for a clinician to build a new therapy session for a patient in accordance with some embodiments; and

FIG. 26 is a flow chart depicting an example implementation of a set of instructions to provide a therapy recommendation in accordance with some embodiments.

While the present disclosure is susceptible to various modifications and alternative forms, specific embodiments have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the disclosure as defined by the appended claims.

### DETAILED DESCRIPTION

The following description is provided as an enabling teaching of a representative set of examples. Many changes can be made to the embodiments described herein while still obtaining beneficial results. Some of the desired benefits discussed below can be obtained by selecting some of the features discussed herein without utilizing other features. Accordingly, many modifications and adaptations, as well as subsets of the features described herein are possible and can even be desirable in certain circumstances. Thus, the following description is provided as illustrative and is not limiting.

This description of illustrative embodiments is intended to be read in connection with the accompanying drawings, which are to be considered part of the entire written description. The drawing figures are not necessarily to scale and certain features of the invention can be shown exaggerated in scale or in somewhat schematic form in the interest of clarity and conciseness. In the description of embodiments disclosed herein, any reference to direction or orientation is merely intended for convenience of description and is not intended in any way to limit the scope of the present disclosure. Relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral,” and the like are to be interpreted relative to one another or relative to an axis of elongation, or an axis or center of rotation, as appropriate. Terms concerning attachments, coupling, and the like, such as “connected” “interconnected,” “attached,” and “affixed,” refer to a relationship wherein structures are secured or attached to one another either directly or indirectly through intervening structures, as well as both movable or rigid attachments or relationships, unless expressly described otherwise. The terms “operatively connected” or “operatively coupled” are such an attachment, coupling or connection that allows the pertinent structures to operate as intended by virtue of that relationship. The term “adjacent” as used herein to describe the relationship between structures/components includes both direct contact between the respective structures/components referenced and the presence of other intervening structures/components between respective structures/components. As used herein, use of a singular article such as “a,” “an” and “the” is not intended to exclude pluralities of the article's object unless the context clearly and unambiguously dictates otherwise.

In various embodiments, a differentially-applied frequency-separated electrotherapy apparatus and method are disclosed for providing therapeutic electric current to a treatment site of a patient. One or more embodiments of the electrotherapy device may include at least two individually generated and amplified oscillating or pulsing alternating currents, of frequencies which differ from each other by as a little as 1 Hz and up to about 300 Hz, wherein the base frequency value of the two frequencies can be between 200 Hz and 500 KHz. The apparatus and method may require at least two electrodes adapted to act as pain site and return electrodes which provide electric current beneath the epidermal or mucous surface of the patient, directly over or next to the source of pain.

In some embodiments, the method of electrotherapy includes providing two individually generated and amplified signals with a frequency difference between them which is applied to one or more pairs of electrodes placed on the body directly over locations of pain and/or over the origin of the pain. According to various embodiments, as will be described in further detail below, since the signals share a common power supply return path, each signal's electrode acts as the return path for the opposing signal. Advantageously, the signals non-linearly mix on polarizable weakly rectifying structures along the current path to evoke a neuro-stimulated pain signal transmission blocking effect by interfering with nerve impulse signal transmission.

In various embodiments, at least one pair of electrodes are placed directly over locations of pain, on or beneath the epidermal or muscular surface of a patient coupled to a generator feeding via the at least one pair of electrodes with two or more oscillating or complex morphology electric currents to a patient. In some embodiments, the respective selected electrode placement locations are opposite one another on the patient's body with a pain site located on a line vector in between the electrodes with the line vector perpendicular to each skin surface on which the electrodes reside. In various embodiments, as described below, the at least one pair of electrodes may be placed directly over a single location of pain. In some embodiments, the currents generated by the at least one pair of electrodes operate at a frequency of at least about 1 KHz and have a current difference between each electrode respectively as little as 1 Hz by up to about 300 Hz. As described in part above, a non-linear action of nerve fiber membranes causes a multiplication of the two independent high frequency signals in a volume of tissue surrounding and beneath each of the at least two electrodes to produce a therapeutic effect in the hemisphere surrounding and beneath each of the at least two electrodes. The multiplication yields a distribution of synthesized sum and difference frequencies among which is a therapeutic low frequency signal that is equivalent to a beat frequency of the signals.

As described in part above, two high frequency electronic wave-forms, or feed signals, are introduced into the body non-invasively through at least one pair of disposable electrodes placed on the skin directly over the pain site, according to some embodiments. In various embodiments, for two locations of pain, each electrode is placed directly over a painful area. In some embodiments, for one location of pain, one electrode is placed directly over a single location of pain, the second electrode may be placed over a bony area which is a comfortable location to receive stimulation.

The multiplication of the two high frequency feed signals (sinewaves) gives rise to a new spectrum of frequencies which are comprised of the following: three high frequency components, a low frequency component, and harmonics on the low frequency component. The three high frequency components include the two high frequency feed signals and the summation of the two high frequency feed signals. The low frequency component is equal to the difference between the high frequency feed signals (or beat frequency). The harmonics on the beat frequency are comprised of frequencies equal to two times (2×) the beat frequency, three times (3×) the beat frequency, four times (4×) the beat frequency, etc. all at declining intensities. The result of the multiplication of the two high frequency feed signals, which are the beat frequency, harmonics and associated high frequency components, form an active electric field within the volume of tissue in the shape of a hemisphere below and surrounding each electrode. The key components of the multiplication of the two high frequency feed signals that affect pain reduction are the combination of the low frequency component and the three high frequency components. The size of the active electric field is defined by the geometry of the electrode, which could be circular, rectangular, a square, a custom shape designed to be placed over a specific body part, or any other shape. The size and shape of the volume of tissue affected can be changed and is dependent upon electrode placement, geometry and materials, as well as the amplitude of the high frequency feed signals.

Embodiments of the present disclosure provide a pain management system that includes a database configured to compile and analyze passive and active patient information utilizing the electrotherapy device and a patient facing application. One or more processors of the database can provide therapy recommendations to the patient in order to more effectively utilize the electrotherapy device, such as changing the intensity of the device or the placement of the electrodes at the treatment site. Embodiments of the present disclosure may also allow for a clinician facing application in the pain management system in order for the clinician to see the status of their patients and to send patients messages and additional therapy recommendations. Overall, embodiments of the present disclosure lead to a more effective use of the electrotherapy device, greater adherence to a patient's therapy plan, and provide valuable insights on the use of the electrotherapy device that will have widespread effects on pharmaceutical development and healthcare in general.

Valuable insights from the use of the pain management system may be used during drug clinical studies to providing pharmaceutical companies with information to accelerate drug development. For example, the electrotherapy device of the present disclosure may be added to a pain drug trial to increase the effectiveness of the drug in combination with the electrotherapy device.

Further, the use of the electrotherapy device of the pain management system disclosed herein may allow for a smaller dose or lower strength pain drug than would be prescribed without the use of the electrotherapy device of the present disclosure. In the alternative, the pain management system may be used to exclusively treat pain without the use of drugs. The insights gained through the use of the pain management system may be used to change and improve treatments/therapy sessions for the patient, inform healthcare providers in real-time of the adherence, frequency, and effectiveness of the pain management system. The insights may also lead insurance companies to change payment decisions for such electrotherapy devices, therapy plans, and treatments.

### Physiological Application

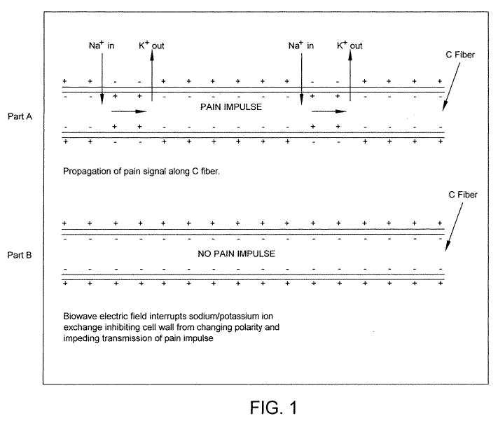

FIG. 1 illustrates the hyperpolarization mechanism of pain reduction according to various embodiments. Pain signals from receptors that are large enough to exceed the trigger threshold for the exchange of sodium and potassium ions across a nerve cell membrane do so through changes in the ion permeability of this membrane. This ion exchange causes a polarity change across and along the cell wall of the nerve fiber affecting the transmission of pain information along certain C type fibers as shown in Part A of FIG. 1. Several mechanisms of action caused by the beat frequency and the three high frequency components discussed above to reduce pain, namely (1) Frequency Conduction Block (also called Hyperpolarization), (2) Gate Control, (3) increased blood flow and (4) Afferent Stimulation, which produces the release of endorphins or other opiate-like analogs.

Frequency Conduction Block. In Part B of FIG. 1, with the low frequency electric field in place, the membranes of C fibers that fall within the electric field are hyperpolarized. As a result, the sodium/potassium ion exchange is inhibited and the cell wall is prevented from changing polarity (from a negative potential to a positive potential) thus impeding the transmission of action potentials. As a result, pain impulses along the C fibers are blocked—similar in action to local chemical anesthesia, except without any deleterious side effects.

A further explanation of the therapeutic Hyperpolarization mechanism is that the resulting beat frequency, its signal morphology, and current densities within the volume of tissue around and below each electrode, causes an alteration in the nerve cell membrane's sodium/potassium ion concentrations or ion exchange kinetics. As a result, the charge polarity of the nerve cell wall is prevented from changing and is therefore unable to transmit pain impulses.

Empirically, the combination of the difference signal along with the three high frequency components does affect the sensory fibers, as some loss of proprioception at the skin as well as induction of hypoesthesia in the region of the active low frequency electrical field occurs about 5 minutes into the treatment, similarly to but not as absolute as a chemical anesthetic. Following a 30-minute treatment, hypoesthesia remains typically for up to 30 minutes post treatment.

Empirically, the combination of the difference signal along with the three high frequency components also affects muscle tissue, which is polarized, in that it holds muscle tissue in tension during the treatment, which results in the patient feeling a deep, smooth sensation from the electrical field which is comfortable and provides for excellent patient compliance using the device.

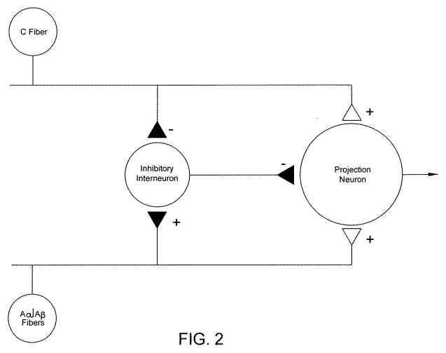

Gate Control. Gate Control focuses on interactions of four classes of neurons in the dorsal horn of the spinal cord as shown in FIG. 2: (1) C fibers which are unmyelinated, (2) Aβ/Aδ fibers which are myelinated, (3) projection neurons whose activity results in the transmission of pain information, and (4) inhibitory interneurons which inhibit the projection neuron, thus reducing the transmission of pain information.

The projection neuron is directly activated by both Aβ/Aδ and C fibers. However, only the Aβ/Aδ fibers activate the inhibitory interneuron. Thus when Aβ/Aδ fibers are stimulated by the combination of the beat frequency along with the three high frequency components from the electric field, the inhibitory interneuron is activated and prevents the projection neuron from transmitting pain information to the brain. The C fiber is left in a state analogous to an open electrical circuit so that transmission of the sensation of pain is suppressed.

Increased Blood Flow. An additional mechanism of action is that the resulting low frequency electrical field that forms beneath and surrounding both electrodes can accelerate any charged species under its influence. This may lead to an increase in local blood flow. Medical studies have shown that proper blood flow is required for the healing of any wound or injury. With the treatment application of the apparatus, there appears to be a concomitant increase in blood flow in the volume of tissue where the electric field is present that accelerates healing. Clinical evidence shows there is also a concomitant increase in range of motion and reduction of stiffness for up to 24 hours following the treatment.

Release of Endorphins or Other Opiate-like Analogs. Afferent Stimulation has been proven in-vivo to provide residual pain relief that can last for up to 24 hours following a 30-minute duration treatment/therapy session. The supporting empirical evidence of the residual pain relief is based on the fact that Afferent Stimulation is responsible for the local release of endorphins, serotonin, enkaphlins, or other opiate-like analogs along with a refractory mechanism involving the membrane itself contributing to the residual pain relief.

### Unique Control and Management Apparatus and Method

According to various embodiments of the present disclosure, the electrotherapy device controls the output of a handheld high frequency neurostimulator for providing a therapeutic treatment inside the body to treat pain and other conditions by utilizing a digital amplifier, feedback control utilizing filters, and other circuitry to provide comfortable treatment to patients. Advantageously, the electrotherapy device described in the present disclosure eliminates electrical spikes and jolts regardless of if the patient is sitting or moving about during the treatment.

One embodiment of the electrotherapy device involves two signals: S1 represents a first signal at a first frequency and S2 represents a second signal at a second frequency. S1 and S2 are linearly independent AC signals. At any given instant one electrode can act as the source of the signal while the other electrode can serve as its return. Due to the AC nature of the signal these roles become reversed as a function of the instantaneous polarity of said signal. The time dependent roles of the electrode vary for the two signals as they are not in phase. It will be appreciated that the effect within the body from the combination of S1 and S2 passing through the body to the respective electrodes produces the pain-relieving effects described above.

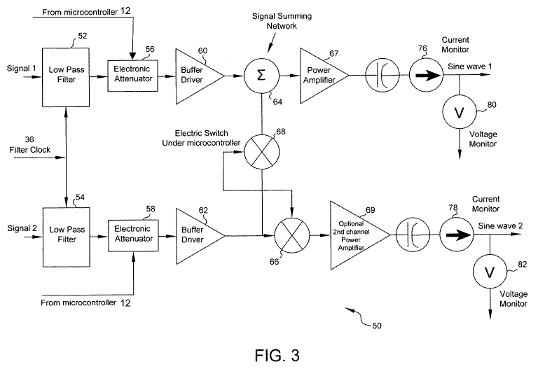

FIG. 3 illustrates output portions of an electrotherapy device in accordance with some embodiments of the present disclosure. More specifically, FIG. 3 depicts a sub-system 50 for converting Signal 1 and Signal 2 to sine wave signals. As discussed above, the output signals of the electrotherapy device need to be as close to a pure sine wave as possible. Signal 1 and Signal 2 are initially logic level square-type waves. These signals are limited to 0.6V amplitude by the transistor limiters. The outputs of these limiters are applied independently to high-order low pass filters 52 and 54. The filter clock 36, if switched capacitor filters are used, output is coupled to each of the filters. These filters suppress the higher order harmonics present in the limited square waves leaving low distortion sine waves at the reference frequencies. These sinusoidal signals are amplified and applied to electronic attenuators (or programmable amplifiers) 56 and 58 (under microcontroller 12 control) to control the level of the signal applied to the power amp stage, discussed below, and ultimately to the patient. The signals are then buffered 60 and 62 and applied to a power gain stage. The power stage consists of one or more amplifiers 67,69 capable of supplying a wide range of voltages into any physiological and electrode load over the frequency ranges used. Depending on the desired level of system integration and/or portability required, this amplifier stage can be either of the linear Classes A or AB₁ or the nonlinear switching Class D type. In various embodiments, use of the Class D amplifier, as discussed in further detail below, provides the efficiency and in turn, minimal heat generation properties, to allow enclosure of the therapeutic device for water resistant properties.

For Class D amplifiers a high-speed comparator varies the pulse width of a switching power transistor (MOSFET type). This modulation is called pulse width modulation and is driven by the original signal's frequency, amplitude and desired gain. The sampling of the reference signal, derived from either a PLL reference or DDS, is sampled at a rate at several orders of magnitude higher than the highest frequency component in said reference. The output of the power transistor is low-pass filtered by a passive LC network to yield the amplified signal. The mode of amplifier operation is particularly attractive since power conversion efficiencies of over 90% can be obtained as opposed to the efficiencies of linear amplifiers which are between 40% to 70%. The microcontroller 12 sets, via electronic switching 68, whether the signals are summed at an amplifier to create the mixed signal or applied individually to the power stage and thereby allows the mixing to take place within the patient's body. Additionally, one or more channels and/or return signal paths can be multiplexed with electronic power switching during zero crossing of the sine wave signals (via processor control). This multiplexing or switching allows multiple electrodes to be fed from the amplifiers or connected to an analog return. This is done to synthesize a larger effective target region on or within the patient. The patient is electrically isolated from leakage to power mains by the isolated plastic housing of the Apparatus and by the use of a battery power supply.

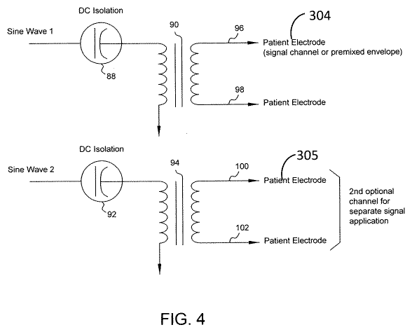

FIG. 4 illustrates the coupling of Sine wave 1 and Sine wave 2 to the electrodes when the apparatus is constructed using around ground referenced (local Apparatus ground) linear power amplifiers in accordance with some embodiments of the present disclosure. The sine wave signal is coupled from the junction of current monitor 76 or 78 and voltage monitor 80 or 82 to a DC isolation capacitor 88 or 92. This capacitor removes any remaining DC component on the sine wave signal. The sine wave signal is coupled to transformer 90 or 94. The output of the transformer 90,94 is coupled to the patient electrodes. One output of each transformer 96 or 100 is coupled to a large signal electrode and the other to a small return electrode 98 or 102. The transformer provides voltage gain and patient/apparatus isolation. With bridged amplifiers or in Class D operation no such transformers are required unless additional voltage gain is needed. In various embodiments, the Dispersive electrode has a much larger surface area contacting the patient than the Pain Site electrode. This size ratio of the Dispersive electrode to the Pain Site electrode is at least 2:1. In some embodiments, the electrodes are the same size and act as both pain site and return electrodes for each other depending on the opposing delivery of the signals.

In some embodiments, a feedback network is disclosed. In various embodiments, the feedback network consists of two functional parts: 1) a circuit (Hardware), that monitors the patient-applied current and possibly voltage and 2) software that determines if the values measured require an output level change (Software). The parameter derived from the current and voltage is the impedance across the patient-applied electrodes. This parameter has been found by studies to be essentially invariant at a given frequency (frequency interval for this device) and over the range of applied potentials used clinically. Further, any impedance change due to a change in patient position essentially disappears when he or she either returns to the position held before the impedance change or after there is an equilibration of blood flow.

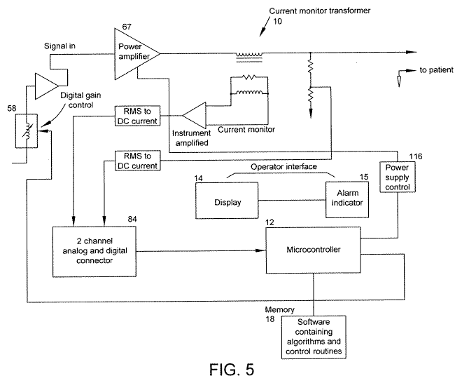

FIG. 5 illustrates the structure of an electrotherapy device according to some embodiments. In various embodiments, a microcontroller 12 supervises the entire operation of the apparatus. The microcontroller 12 is responsible for interpreting operator commands and for displaying system status on the display panel 14. Additionally, the processor controls the frequencies of the signal sources, their levels and compensates for any variation in system load. This last function is important since changes in patient electric load can affect the signal level and the perceived sensation of the apparatus effect. The microcontroller 12 uses feedback to control signal levels by comparing the immediate electrical load to previously “learned” characteristic rules for a particular patient. The microcontroller 12 provides input to the electronic attenuator 58. Additionally, the microcontroller 12 receives operation instructions from software containing algorithms and control routines stored in memory 18. In various embodiments, memory 18 may be pre-programmed by an operator. The microcontroller 12 provides instructions to various portions of the signal generation system. The signal system generates two signals. In some embodiments, microcontroller 12 is also responsible for displaying alarms and indications via an indicator unit 15. In some embodiments, this includes an LED display unit having different colors. By way of example, the indicator unit 15 may display Green for indicating battery strength or charge level of the portable unit. Other parameters may identify Bluetooth® capability, signal intensity, treatment time, and/or indicate errors or aid in troubleshooting. One of ordinary skill in the art will appreciate that the indicator unit 15 may display various visual indicators useful to a patient for displaying alarms and operations of the electrotherapeutic unit.

The microcontroller supervises the operation by adjusting the electronic attenuator 58 for the apparatus. As described above, the signals from above are buffered 60 and 62 and applied to a power gain stage. The power stage consists of one or more amplifiers 67, 69 capable of supplying a wide range of voltages into any physiological and electrode load over the frequency ranges used. The second class of amplifiers, which also improves performance in a portable system, is that of Class-D.

As described above, there are several ways of generating and amplifying signals. All methods rely on individual oscillators and amplifiers. Class AB₁ amplification is a well-known method for amplifying sinusoidal signals. In the present disclosure the input to these amplifiers are controlled-amplitude sinusoidal signals of differing frequencies. Regulation of the output signal, as a function of load impedance, is achieved by the close-looped feedback network which also can either alter the gain of the power amplifier or the amplitude of the power amplifier's input signal.

Another method uses Class D switching amplifiers. There are two ways these amplifiers can be used to generate the signals. In one method pulse width modulated signals, representing the two frequencies is generated by a microcontroller 12. The width of the pulses defines the amplitude of the final signals and the rate of the pulse packet defines the frequency. These pulse packets drive a set of field effect switching transistors. The output of these transistors is low-pass filtered, reconstructing the sinusoidal signal of the desired amplitude. The second method uses a comparator, connected to a reference sinusoidal signal of set amplitude and a triangular ramp signal. The output of the comparator is a pulse width modulated signal that drives the same circuit, as mentioned above, to generate the output signal. Regulation of the output signal can be achieved by a feedback loop from the output to a summing circuit at the input or monitoring the output using an analog-to-digital circuit on the system's microcontroller 12. The microcontroller 12 can use the digital values of the changes in the output signal, due to changes in load impedance, to adjust the pulse width modulation signal to compensate for these variations.

The unique third method is one derived from high-efficiency radio frequency amplifiers—Class E. Class E is a switching amplifier where a power MOS field effect transistor is driven by a square wave signal whose repetition rate corresponds to the desired output frequency. The amplified pulse is bandpass-filtered recreating an amplified sinusoidal signal. The amplitude of the signal is set by the power supply voltage level. Regulation of the output is achieved by sampling the output signal and using it to control the power supply voltage level to maintain fixed output signal amplitude independent of load impedance. The regulation circuit can be realized by direct hardware feedback or by using the microcontroller's 12 analog-to-digital converter to measure the output amplitude and using the difference between desired amplitude and actual amplitude to set the control voltage on the power supply.

Advantageously, the ability to regulate the output of a digital amplifier into a dynamic load makes for a much more comfortable smooth treatment sensation as the patient moves during treatment. This ultimately results in excellent patient compliance using the device. Regulation of the output signal can be achieved by a feedback loop from the output to a summing circuit at the input or monitoring the output using an analog-to-digital circuit on the system's microcontroller. The microcontroller can use the digital values of the changes in the output signal, due to changes in load impedance, to adjust the pulse width modulation signal to compensate for these variations.

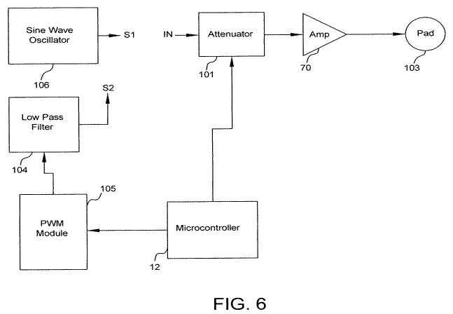

FIGS. 6-8 illustrate the general block structures of an electrotherapy device in accordance with some embodiments of the present disclosure. In FIG. 6, according to some embodiments, S1 represents a sine wave reference signal generated by an analog oscillator 106. S2 represents a sine wave reference signal which is derived from low-pass filtered 104 pulses generated by the pulse width modulation (PWM) 105 module within the microcontroller 12. These are two possible ways of producing the reference signals. Attenuator 101 controls the amplitude of the reference sine wave which is fed to a class AB power amplifier 70. The output of the power amplifier 70 is applied to the patient-connected electrode 103. According to some embodiments, each channel requires (either 106 or 104), 101, 70, 12 and 103.

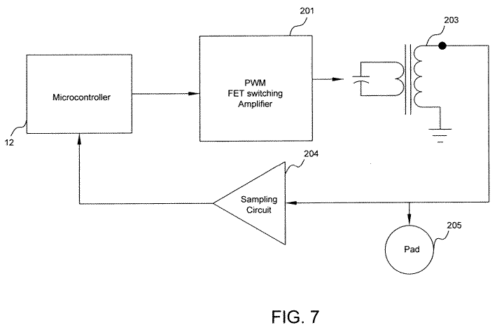

In FIG. 7, according to various embodiments, microcontroller 12 generates a PWM signal where the relative widths of the pulses control the ultimate amplitude of the final signal. A MOSFET transistor bridge switching network 203 is driven by the PWM signal described above. The output of this bridge is a large-signal replica of the original PWM signal—Class D. This signal is passed to a low-pass filter network 203 with a cutoff frequency much lower than the pulse rate of the PWM signal. The transformer supplies voltage gain to enable the use of low voltage power supplies and low voltage monolithic or discrete device class D amplifiers. Two forms of feedback, for signal regulation, can be used: (1) A direct feedback network in the loop between the output of the switching MOSFETs to the input or (2) Using the microcontroller's 12 analog-to-digital converter to sample 204 the analog output voltage and correct this voltage by dynamically varying the PWM signal. Each channel requires 201, 203, 204 and 205.

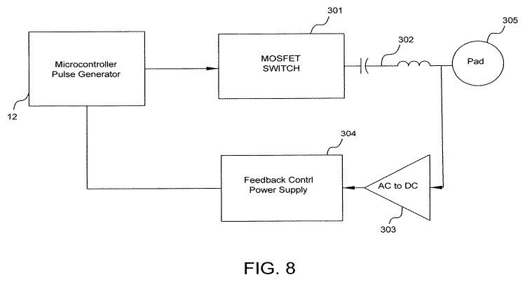

In FIG. 8, according to various embodiments, a Class E embodiment is disclosed. Class E is a switching amplifier where 50% duty-cycle pulses drive a power switch. The pulse repetition rate is at the frequency of interest. Microcontroller 12 generates the logic-level pulses. This signal drives a MOSFET power 301 transistor whose output swings between the power supply rail and near ground. This output signal is applied to an inductor/capacitor network 302 resonant at the frequency of interest. This signal is applied to the patient-connected electrode 305. Output amplitude is entirely set by the power supply rail voltage 304. The output signal is sampled and converted to a DC correction voltage 303. This voltage is used to trim the power supply voltage thereby regulating the output signal. Each channel individually requires 301, 302, 303, 304 and 305.

Class E amplifiers are characterized by simple design, construction and relatively high efficiency (>=90%). Our therapeutic signal difference of around 122 Hz can be delivered over a band of frequencies ranging from around 1 KHz to 30 KHz. As the frequency rises the body-load impedance drops. Therefore, for a given delivered power a lower output voltage is required. Class E amplifiers require two amplifier channels each separately applied to one of the two electrodes. The second electrode acts as the return path for each signal. Class E amplifiers are pulse-switched tuned-output devices where the load impedance is matched to the tuned output network of the amplifier. The design of the amplifiers as disclosed according to some embodiments requires that each amplifier be tuned to some mid-band frequency (e.g., 10 KHz and 10.122 KHz) at the average body load impedance. The operational voltage is set by the amplifier's MOSFET drain voltage. If the patient load varies it will be reflected in the measured applied voltage and current. These voltages and currents are monitored by the system microcontroller 12. The contents of look-up tables, indexed by the desired voltage and expected current, are compared to the drain voltage and the measured voltage and current. The error in expected and measured voltage and current are used by an algorithm to determine what change in operating frequencies would be required to return the output signal to its proper power density. Since, as indicated above, we have a fairly broad available frequency range it should be possible to dynamically correct for the impedance mismatch and apply the proper power to the patient load.

### Transformer

For both safety and economic reasons, it is desirous to operate the device's power amplifier section at lower output voltages. In terms of safety, the use of low voltage power amplifiers guarantees that a harmless D.C. voltage level would be applied to the patient if the D.C. isolation mechanism should fail. Additionally, the use of lower supply rails lessens the complexity and cost of the power amplifier's power supplies and greatly broadens the number and types of power amplifier topologies and/or devices that can be used. This allows for more choice in determining the best power amplifier for a given price and performance. In the device, transformers can supply either D.C. isolation and/or voltage gain. In one embodiment, a high coupling toroidal transformer was used to increase the device output voltage by a factor of 2.4. This kept the power supply design simple and inserted a magnetic isolation barrier between the patient and the device. In another embodiment, as discussed in more detail below, an autotransformer configuration is used to boost the output voltage from 6 V RMS to 36 V RMS. However, the inherent losses and non-linear responses found with any transformer causes its output voltage to vary as a function of the load it is connected to. This failure-to-follow or poor regulation can and does lead to patient discomfort. In order to take advantage of a transformer's voltage gain it is necessary to compensate for poor regulation.

Poor regulation can be overcome via two methods: (1) Electronically—where a sample of the output controls the gain of the output circuitry; and (2) Utilizing the microcontroller 12—where a sample of the output is converted and used by the microcontroller 12 to determine a correction to the setting of the digital intensity control.

For the configuration where the transformer has isolated primary and secondary windings, the output is sampled and returned to the amplifier section through an isolation amplifier. This is required in order to maintain the D.C. isolation barrier created by the transformer. The output of the isolation amplifier is used to either vary the bias on a transconductance amplifier or the resistance of an attenuator which controls the gain of the device's preamplifiers or power amplifier directly, in response to deviations in the output signals relative to a reference. For the autotransformer configuration, no isolation amplifier is used since this transformer-type is inherently non-isolating. In this case capacitors are used to isolate the D.C. from the output. Regulation for this transformer output is maintained by connecting the transformer primary tap or an attenuated signal developed from the high voltage tap back to the inverting input of the power amplifier. This closes the amplifier loop thereby dynamically compensating for the transformer's non-ideal behavior.

### Safe Operating Limits

Paramount to any medical electrical device is the prevention or discontinuation of device's operation when it encounters an unsafe condition. For the electrotherapy device we have developed, the major unsafe condition arises when the applied current causes a rise of skin temperature above 41° C. causing a thermal burn. Another condition, which is more unpleasant than dangerous, is when the output voltage abruptly changes as a function of load change. This is perceived by the patient as a surge-like feeling. This condition is normally not associated with an increase of skin current density and as such cannot cause injury.

There are two methods which have been used to ameliorate the burn-mode of device operation. One method uses the microcontroller 12 and its software to determine if the current flow exceeds a pre-programmed limit. The output current is sampled either by a small-valued series resistor or a resistor terminated current transformer. The analog level which represents the output current is converted to a digital value and compared continuously with the preset limit. When this limit is exceeded the software turns off the power amplifier(s) or their power supplies and signals the user to the over-current condition.

The second method of safe operational control also uses a measure of the output current or a measure of the load impedance as determined from this current and applied voltage. Current monitoring is affected as with the limit control above. Voltage monitoring is performed by sampling the output voltage and converting it to a digital representation of the RMS applied voltage. Software uses these values to determine if operation is exceeding safety guidelines. For example, a drop in load impedance increases the output current. Impedance values derived from low output-level startup current and voltage values are used to determine impedance measures. An algorithm sets the allowed current limits for a given output level. If device operation falls outside of these limits, for a predetermined period, the device can shut down the device or the ability to increase signal intensity can be disabled. The use of an operational-limit algorithm and time measure is critical since there can be situations (for example, output settling or momentary electrode condition changes) where operation falls outside certain limits but are not a reflection of a device failure or other unsafe condition. Further, dynamic lowering of the device output level is used when for a given intensity the impedance changes outside of predetermined limits for a given period. This mode of operation is used to lessen or eliminate the chance of a burn when the power density rises above guideline limits. The operator can still bring down the intensity and need not stop operation as long as the maximum allowed current is never exceeded. Normal device operation is restored when the measured impedance returns to within pre-determined operational limits. If this fails to happen within a predetermined elapsed time the device is disabled, and the condition is indicated to the operator.

### Timer

According to various embodiments, a timer, which can be auto-loaded with a default treatment time or have the treatment time set by the operator, is initialized and maintained by the device's system software. This timer has several uses. It shuts off the device at the end the elapsed treatment time and it acts as a reference for the safe-operation-limits software to help determine whether a time-dependent excursion outside of normal impedance boundaries is interpreted as a failure or transient event. This could include limiting the number of treatments a patient can receive within a pre-determined period. The timer can also be used to change the device output intensity as a function of a pre-loaded time-sequenced treatment protocol. The amount of aggregate treatment time accumulated by the device is updated by the timer at the end of each treatment session. This information is used to determine when battery replacement or other service procedures should be performed.

### Autotransformer

It is useful if the operating voltage of the output power amplifier could remain low. This lessens losses in the switching power supply that increase as the voltages needed rise. Additionally, higher voltage amplifiers are more expensive and usually physically larger. In various embodiments, one method to achieve voltage gain is by using a transformer. Typical transformers have a primary winding and a secondary winding. They offer voltage or current gain while isolating the input circuit from the output circuit. Unfortunately, there are losses associated with the core of the transformer, the winding resistance and imprecise coupling (magnetic) between the primary and secondary winding. One way to utilize the voltage gain capabilities of a transformer is through the use of the autotransformer configuration. Here the primary and secondary share the same winding. For voltage gain assume that the input signal, in closed feedback loop with the output amplifier, is applied to N turns of wire wrapped around a ferromagnetic core (ideally a toroid) the secondary winding is just a continuation of the primary winding (electrically the same wire). To get twice the voltage from the secondary the winding is continued for another N turns on the same core. The output is taken from the end of the secondary winding. In this configuration there is tighter magnetic coupling and good output regulation (as opposed to what is found with isolated primary and secondary windings). Additionally, the autotransformer is cheaper, electrically better and smaller than a normal transformer. If desired, the output at the secondary can be attenuated and if need be phase-shifted and used to close the loop of the power amplifier. The attenuation is necessary to maintain the amplifier's differential input voltages close in value as the feedback loop requires.

### Construction

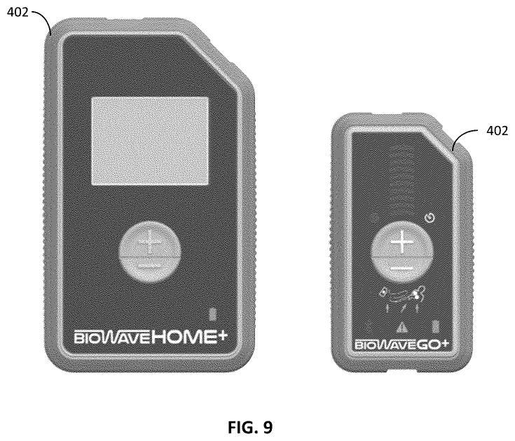

FIG. 9 illustrates two different exemplary electrotherapy devices 402 in accordance with some embodiments. According to various embodiments, the electrotherapy device 402 includes an option for physically manipulating the intensity of the treatment. In some embodiments, the electrotherapy device 402 includes a communications unit for communicating with a client device to adjust the parameters remotely. For example, the electrotherapy device 402 may be operated remotely using a client device connected via Bluetooth® or Wi-Fi communications. It should be appreciated to one of ordinary skill in the art that a computing device may remotely connect to the electrotherapy device 402 in various ways for operation. In some embodiments, the electrotherapy device 402 may include an angled female port for connecting the electrodes. The angled port advantageously permits ease of access and wearable functionality for the electrotherapy device. In various embodiments, the angled port includes a depression for recessing the connection of the electrodes. In some embodiments, the recessed port includes a plurality of indentations configured to receive a cable attached to the male connector such that the cable is located against the side edges of the substantially rectangular device when the male connector is inserted into the female port. In some embodiments, the electrotherapy device 402 may include a display to therapy session information, such as the duration of a therapy session/treatment, the connection status with a computing device, battery status, intensity level, and/or warnings just to give a few examples.

### Pain Management System

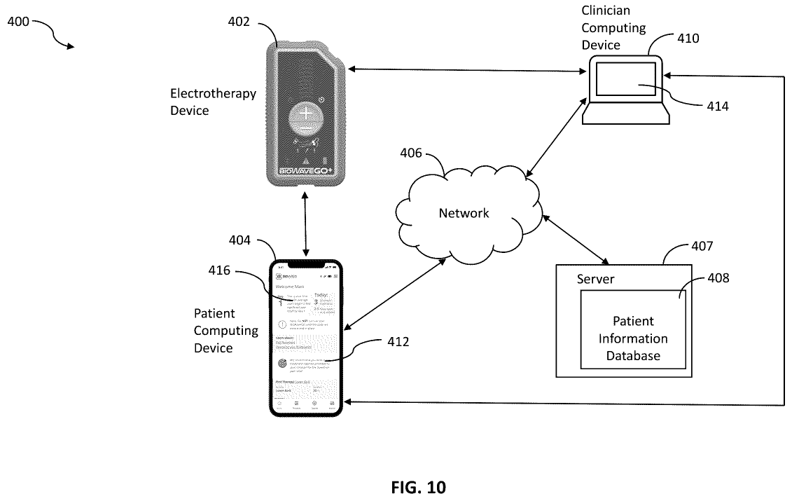

FIG. 10 illustrates one example of a pain management system 400 in accordance with some embodiments. Embodiments of the pain management system 400 includes an electrotherapy device 402, a patient computing device 404, network 406, server 407, patient information database 408, and a clinician computing device 410. The electrotherapy device 402 may be any medical device capable of communicating with and being operated by a patient computing device 404. In some embodiments, the electrotherapy device 402 is the electrotherapy and neurostimulation medical device disclosed in U.S. Patent Application Publication No. 2020/0121925 incorporated by reference herein in its entirety. The electrotherapy device 402 may be prescribed by a clinician (e.g., physician, therapist, specialist, etc.) to be used in a therapy plan. A therapy plan may include the body part targeted for treatment, the duration of a therapy session, intensity (e.g., maximum, average, and ramp up/down during the therapy session), and length of the overall therapy plan (e.g., days, months, weeks, years, etc.). The individual therapy sessions where the user of the electrotherapy device 402 is actually using the device may be the same throughout the therapy plan or may be altered with more, less, and/or different types of therapy sessions based on the user's response to the therapy plan. It shall be noted that “user” and “patient” are interchangeable with regards to the user of the patient computing device 404 and/or electrotherapy device 402.

The patient computing device 404 of the pain management system illustrated in FIG. 10 may be configured to run and display a patient facing application 412. The patient facing application 412 may also be capable of communicating with and operating the electrotherapy device 402 through by wired or wireless communication. The patient computing device 404 may also be configured to communicated and transfer patient information 416 compiled in the patient facing application 412 to a patient information database 408 and/or a clinician computing device 410 through network 406. In some embodiments, the network 406 may be a cellular or other internet based network capable of transmitting patient information 416 between the patient computing device 404, patient information database 408, and clinician computing device 410.

Continuing to refer to FIG. 10, the patient information database 408 may be configured to store the patient information 416 and analyze the patient information 416 through the use of algorithms, lookup tables, machine learned models, and/or artificial intelligence models. After analysis, the patient information database 408 may then transmit at least one therapy recommendation to a user of the patient computing device 404 through the patient facing application 412. In some embodiments, the therapy recommendation may be a suggestion to change how a user uses the electrotherapy device 402. In other embodiments, the therapy recommendation may automatically adjust a user's therapy session or therapy plan to optimize the use of the electrotherapy device 402 to improve the user's outcome. The clinician computing device 410 may also have a clinician facing application 414 in order to display patient information and provide a user interface for the clinician to communicate with their patients. In some embodiments, the patient facing application 412 and clinician facing application 414 may run on their respective computing devices with operating systems such as Apple iOS, Apple macOS®, Apple iPadOS®, Google Android™, and Microsoft Windows® just to give a few examples. One of ordinary skill in the art will appreciate that any available operating system may be used to run the patient facing application 412 and clinician facing application 414.

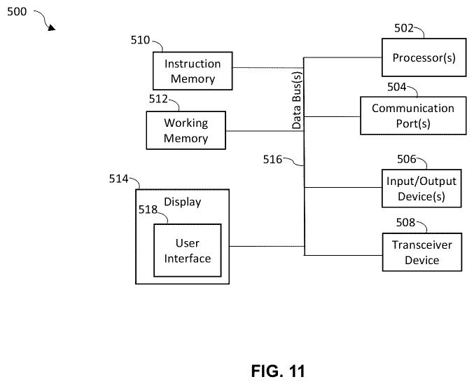

FIG. 11 is an example computing device 500 of a pain management system 400 in accordance with some embodiments. The computing device 500 can be employed by a disclosed system or used to execute a disclosed method of the present disclosure. Computing device 500, such as the patient computing device 404, server 407, and/or clinician computing device 410, can implement, for example, one or more of the functions described herein. It should be understood, however, that other computing device configurations are possible. Computing device 500 may be a smartphone, desktop, laptop, or other type of computing device.

The computing device 500 can include one or more processors 502, one or more communication port(s) 504, one or more input/output devices 506, a transceiver device 508, instruction memory 510, working memory 512, and a display 514, all operatively coupled to one or more data buses 516. Data buses 516 allow for communication among the various devices, processor(s) 502, instruction memory 510, working memory 512, communication port(s) 504, and/or display 514. Data buses 516 can include wired, or wireless, communication channels. Data buses 516 are connected to one or more devices.

The processor(s) 502 can include one or more distinct processors, each having one or more cores. Each of the distinct processors can have the same or different structures. Processor(s) 502 can include one or more central processing units (CPUs), one or more graphics processing units (GPUs), application specific integrated circuits (ASICs), digital signal processors (DSPs), and the like. The processor(s) 502 can also be configured to perform a certain function or operation by executing code, stored on instruction memory 510, embodying the function or operation of the pain management system 400 and discussed herein. For example, processor(s) 502 can be configured to perform one or more of any function, method, or operation disclosed herein. Processor(s) 502 may perform operations necessary to carry out the functions of the patient computing device 404, server 407 and/or clinician computing device 410.

Communication port(s) 504 can include, for example, a serial port such as a universal asynchronous receiver/transmitter (UART) connection, a Universal Serial Bus (USB) connection, or any other suitable communication port or connection. In some examples, communication port(s) 504 allows for the programming of executable instructions in instruction memory 510. In some examples, communication port(s) 504 allow for the transfer, such as uploading or downloading, of data.

The input/output devices 506 can include any suitable device that allows for data input or output. For example, input/output devices 506 can include one or more of a keyboard, a touchpad, a mouse, a stylus, a touchscreen, a physical button, a speaker, a microphone, or any other suitable input or output device.

The transceiver device 508 can allow for communication with network 406, such as a Wi-Fi network, an Ethernet network, a cellular network, or any other suitable communication network. For example, if operating in a cellular network, transceiver device 508 is configured to allow communications with the cellular network. Processor(s) 502 is operable to receive data from, or send data to, a network via transceiver device 508.

The instruction memory 510 can include an instruction memory 510 that can store instructions that can be accessed (e.g., read) and executed by processor(s) 502. For example, the instruction memory 510 can be a non-transitory, computer-readable storage medium such as a read-only memory (ROM), an electrically erasable programmable read-only memory (EEPROM), flash memory, a removable disk, CD-ROM, any non-volatile memory, or any other suitable memory with instructions stored thereon. For example, the instruction memory 510 can store instructions that, when executed by one or more processors 502, cause one or more processors 502 to perform one or more of the operations of the pain management system 400.

In addition to instruction memory 510, the computing device 500 can also include a working memory 512. Processor(s) 502 can store data to, and read data from, the working memory 512. For example, processor(s) 502 can store a working set of instructions to the working memory 512, such as instructions loaded from the instruction memory 510. Processor(s) 502 can also use the working memory 512 to store dynamic data created during the operation of computing device 500. The working memory 512 can be a random access memory (RAM) such as a static random access memory (SRAM) or dynamic random access memory (DRAM), or any other suitable memory.

Display 514 is configured to display user interface 518. User interface 518 can enable user interaction with computing device 500. In some examples, a user can interact with user interface 518 by engaging input/output devices 506. In some examples, display 514 can be a touchscreen, where user interface 518 is displayed on the touchscreen. In some embodiments, the user interface 518 may allow a user of a patient computing device 404 to use and interact with the patient facing application 412. The user interface 518 may also allow a user of a clinician computing device 410 to use and interact with the clinician facing application 414.

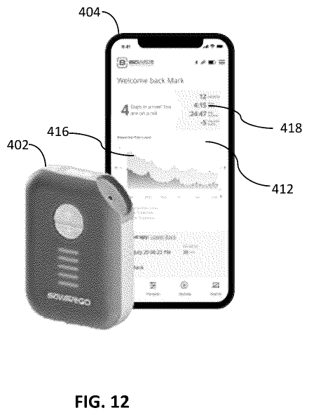

FIG. 12 illustrates wireless communication between an electrotherapy device 402 and a patient computing device 404 in accordance with some embodiments. The electrotherapy device 402 is capable of wired and/or wireless communication with the patient computing device 404. For example, the electrotherapy device 402 may be able to communicate with the patient computing device 404 either through a wire connected to the electrotherapy device 402 and the patient computing device 404, or wirelesslessly through Wi-Fi or Bluetooth® communication. The patient computing device 404 is adapted to run a patient facing application 412, giving a patient the ability to operate the electrotherapy device 402, and also view and input patient information 416. The patient facing application 412 may be configured to passively and actively store and compile patient information 416.

In some embodiments, passive patient information 416 may come from the electrotherapy device 402 (hardware and firmware), which is communicatively coupled to the patient computing device 404. For example, the patient facing application 412 may collect and store patient information 416 and/or electrotherapy device 402 data before, during, and after a therapy session. Examples of passive patient information 416 collected and stored by the patient facing application 412 may include therapy session information, such as the electrotherapy device 402 serial number, duration of a therapy session, date/time of a therapy session, electrotherapy device 402 setting changes (e.g., intensity from 0%-100%, maximum intensity, minimum intensity, mean intensity, voltage, current power, and/or impedance) just to give few examples. Other passive patient information 416 collected and stored by the patient facing application 412 may include electrotherapy device 402 battery use behavior, such as the charge/discharge usage patterns, current charge status, battery temperature, and battery health (e.g., indications of aging). Another category of passive information collected and stored by the patient facing application 412 may include electrotherapy device 402 usage, orientation, and temperature, such as the processor temperature, circuit board temperature, ramp/curve of intensity or max intensity reached, all device button presses and holds and any device button usage patterns.

Other passive patient information 416 collected and stored by the patient facing application 412 may include errors or loss of connection issues, such as lead wire to the electrotherapy device 402 connection, lead wire to the electrodes connection, electrodes to body connection, and/or wireless communication (e.g., Wi-Fi or Bluetooth®) connection issues. Finally, the patient facing application 412 may also collect and store the motion of the user of the electrotherapy device 402 during a therapy session through an accelerometer on the circuit board of the electrotherapy device 402 and optionally the GPS data of a user of the electrotherapy device 402 through the patient computing device 404 and the patient facing application 412. The above passive patient information 416 is provided as an example. One of ordinary skill in the art would appreciate that other passive patient information 416 may be collected by the electrotherapy device 402 and/or the patient facing application 412 to be stored by the patient facing application 412.

The patient facing application 412 may also collect and store active patient information 416 from a user of the patient facing application 412. For example, the patient facing application 412 may collect and store patient information 416 based on what the user of the patient computing device 404 enters into the patient facing application 412. Examples of active patient information 416 may include electrode location placement, a pre-therapy session pain score, a post-therapy session pain score, other concomitant treatments, average duration of pain relief after a therapy session, an activities of daily living (ADL) score, the effect on pain medication consumption, any quality of life improvement, mood improvement, and activity duration such as if the user is sleeping or sitting longer or if the user is walking further than before use of the electrotherapy device 402. The above active patient information 416 are provided as examples. One of ordinary skill in the art would appreciate that other active patient information 416 may be entered into the patient facing application 412 to be stored by the patient facing application 412.

In some embodiments, the patient computing device 404 may be configured to communicate with a patient information database 408 located on server 407 as illustrated in FIG. 10 through the patient facing application 412. For example, the patient facing application 412 may passively or actively transmit the patient information 416 (active and passive) to a cloud based, Health Insurance Portability and Accountability Act (HIPAA) compliant, patient information database 408 for storage and analysis. In some embodiments, the patient information database 408 is configured to analyze the patient information 416 from a first patient and provide one or more therapy recommendations 418. In a non-limiting example, therapy recommendations 418 can include information related to optimizing the use of the electrotherapy device 402 which can further improve the patient's outcome. The optimization can be based on at least one algorithm, lookup table, machine learned model, or artificial intelligence model processed on server 407 by one or more processors 502. The analysis by the patient information database 408 may be based on the first patient's patient information 416 or may be based on an analysis of all of the patient information 416 stored on the patient information database 408. For example, the patient information database 408 may analyze the patient information 416 (active and passive) of a first user of an electrotherapy device 402 and provide a therapy recommendation 418 such as a change in electrode placement, length of a therapy session, maximum intensity attained by the electrotherapy device 402 just to give a few examples. The therapy recommendations 418 may be transmitted by the patient information database 408 to a user of a patient computing device 404 through the patient facing application 412.

In some embodiments, the therapy recommendations 418 may be displayed to a user of the patient computing device 404 for their review and approval. For example, the therapy recommendation 418 provided by the patient information database 408 may display a change to the duration of the therapy session and/or intensity (e.g., maximum, average, ramp up/down, etc.) of the therapy session for the user to either accept, deny, or make changes to. In other embodiments, the therapy recommendations 418 may automatically adjust a user's future therapy session and/or therapy plan without any action by the user. For example, the therapy recommendations 418 may be transmitted from the patient information database 408 and ingested by the patient facing application 412 to adjust some or all of the parameters (e.g., duration, frequency, intensity, etc.) of the user's future therapy sessions and/or therapy plans. In even further embodiments, the therapy recommendations 418 may include some changes to the parameters of the user's future therapy sessions and/or therapy plans that need to be manually accepted, denied, or altered by the user while other changes may be accepted automatically without user intervention.

In some embodiments, the patient information 416 stored and analyzed by the patient information database 408 may be used during clinical studies that can be used to accelerate drug development by giving the pharmaceutical companies insights on pain management. The patient information 416 stored and analyzed by the patient information database 408 may also provide valuable insights to insurance companies, hospitals, and/or healthcare systems that demonstrate the effectiveness and lower cost of the electrotherapy device 402 compared to drugs, surgery, and/or other treatment methods. Some examples of the valuable insights may include therapy plan adherence, usage vs. pain metrics, QoL improvements, ADL improvements, changes in pain medication after electrotherapy device 402 use, mood improvement, and changes in activity duration just to give a few examples.

In some embodiments, the patient information database 408, as described above, may use algorithms, lookup tables, machine learned models, and/or artificial intelligence models in order to optimize the use of the electrotherapy device 402 and improve patient outcomes. For example, the patient facing application 412 may actively and/or passively transmit patient information 416 to the patient information database 408 to be analyzed, through the algorithms, lookup tables, and or machine learned models, and provide a user of the electrotherapy device 402 a unique and personalized therapy recommendation(s) 418 on how or when to use electrotherapy device 402 to optimize the use of the electrotherapy device 402 and improve the user's outcome. Some examples can include the patient information database 408 providing a therapy recommendation 418 to increase the duration of the therapy session or to raise the level of max intensity during the therapy session. This therapy recommendation 418 may be based on the passive and/or active patient information 416 from a first user, which is then compared to all the other users' patient information 416 stored on the patient information database 408. So a therapy recommendation 418 of an increase in therapy session duration or maximum intensity attained during the therapy session may be based on the passive and active patient information 416 from other users of the electrotherapy device 402 with the same injury and optionally around the same age and build as the first patient. This example therapy recommendation 418 to increase the therapy session duration or maximum intensity attained during a therapy session is provided for illustration only. One of ordinary skill in the art would appreciate other ways, metrics, or information to use to provide unique and personalized therapy recommendations 418 through the use of a patient information database 408.

In an example, patient A with knee pain is reporting only a two point (on a scale of 1-10) reduction in pain through the use of the electrotherapy device 402 and the patient information 416 collected from the patient identifies that the maximum intensity reached during the therapy session is 40%. The patient information database 408 will evaluate patient A's patient information 416 and compare to all of the other patients with knee pain. If the patient information database 408 determines that other patients with knee pain are reporting a four point reduction in pain that have a faster ramp up in intensity and a maximum of 60%, the patient information database 408 may provide a therapy recommendation 418 to patient A to ramp up the intensity faster and reach a higher maximum intensity. The patient information database 408 may also provide a therapy recommendation 418 in increasing the frequency of therapy sessions in a given day or week. Other examples of therapy recommendations include a change in electrode placement, decrease in intensity ramp, decrease in maximum intensity reached, decrease in therapy session frequency, an increase/decrease in therapy session duration, increase/decrease in patient motion during the therapy session, and/or adding a concomitant treatment (e.g., pain relief drug, cold therapy, heat therapy, exercise, yoga, stretching, etc.) to a patient's therapy plan.

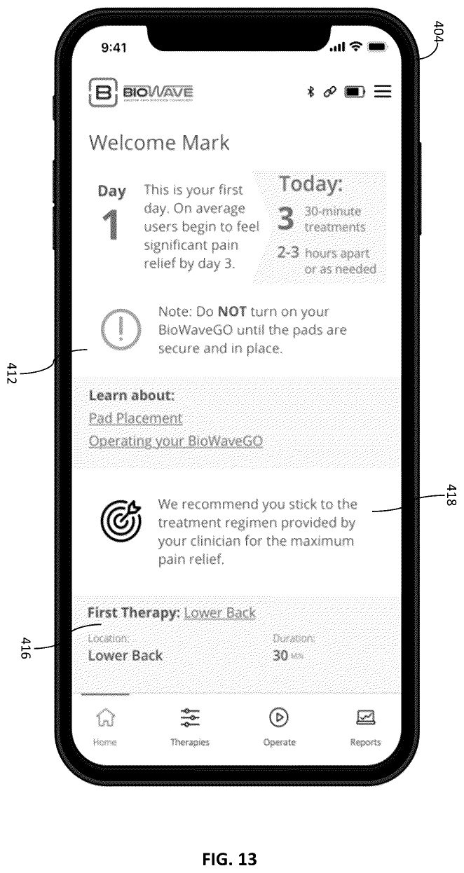

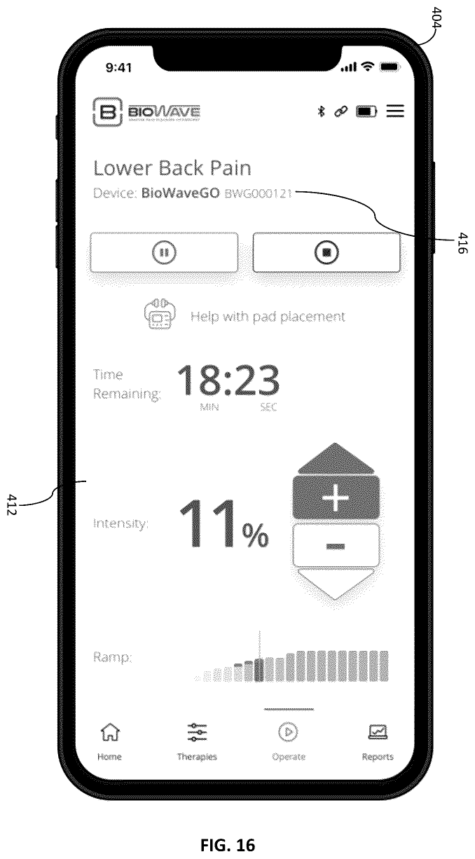

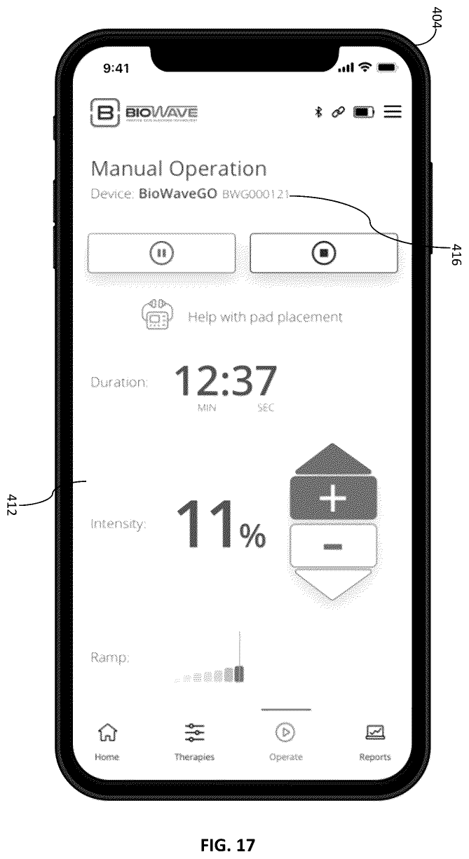

Referring now to FIGS. 13-19, which are different views of a patient facing application 412 in accordance with some embodiments. The patient facing application 412 is configured operate on patient computing device 404 and allow a user of the patient computing device 404 to view therapy plan information. Such therapy plan information may include the active and passive patient information 416 collected by the patient facing application. The therapy plan information may also include previous therapy sessions, a recommended therapy plan, graph of reported pain level (pre-treatment and post-treatment) over a plurality of days or therapy sessions, pictures of proper electrode placement, and directions/tips for how to use the electrotherapy device 402 just to give a few examples.

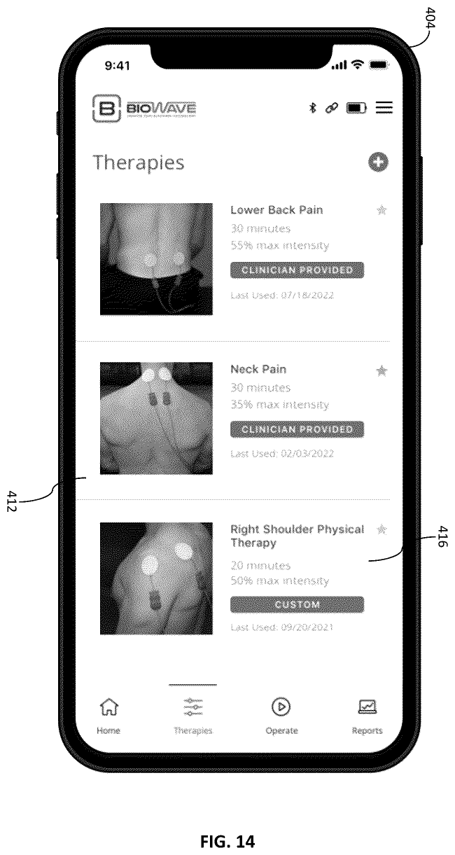

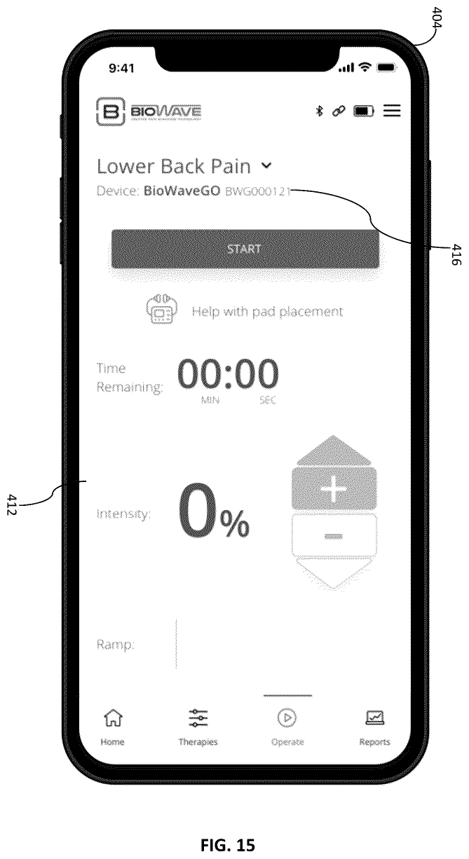

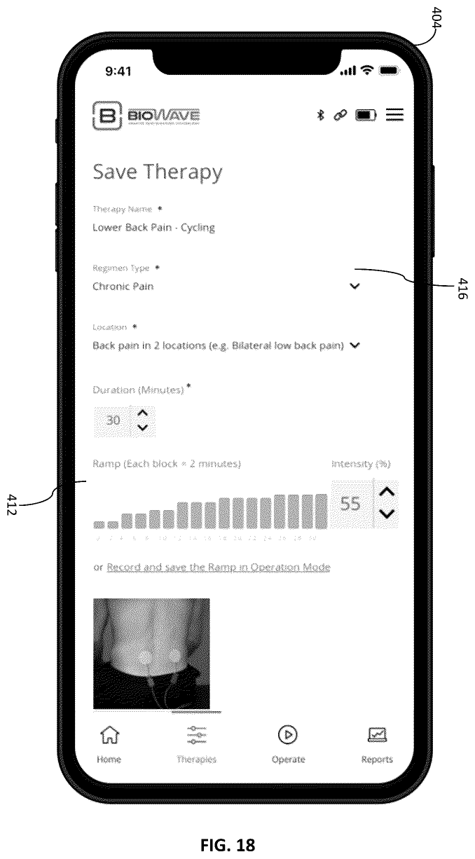

In some embodiments, the therapy recommendation 418 may include the number and duration of the therapy sessions as illustrated in FIG. 13. The patient facing application 412 may also be configured to display previous therapy sessions, including pictures of proper pad placement as illustrated in FIG. 14. The user of the patient facing application 412 will also be able to operate the electrotherapy device 402 either manually or by running a pre-programmed therapy session. For example, a user of the patient facing application 412 may be able to manually start, stop, pause, and adjust the intensity during the therapy session as illustrated in FIGS. 15-17 The patient facing application 412 may also be configured to automatically run a pre-programmed therapy session or recommended therapy session once the user selects the desired therapy session. In this scenario, the patient facing application 412 will adjust the intensity and stop the electrotherapy device 402 according to the selected therapy session plan. The patient facing application 412 may also be configured to allow the user to create, edit, or delete therapy sessions. For example, the patient facing application 412 will allow the user to create a therapy session from scratch and give the user the option to select the body part, the duration, the maximum, mean, and ramp up or down in intensity for the therapy session as illustrated in FIG. 18. After the user creates the therapy session, the user can save the therapy session and take a picture of the electrode placement. An icon with the name of the treatment, such as “Left Should Treatment” may be created to be recalled for later use according to the prescribed therapy plan.

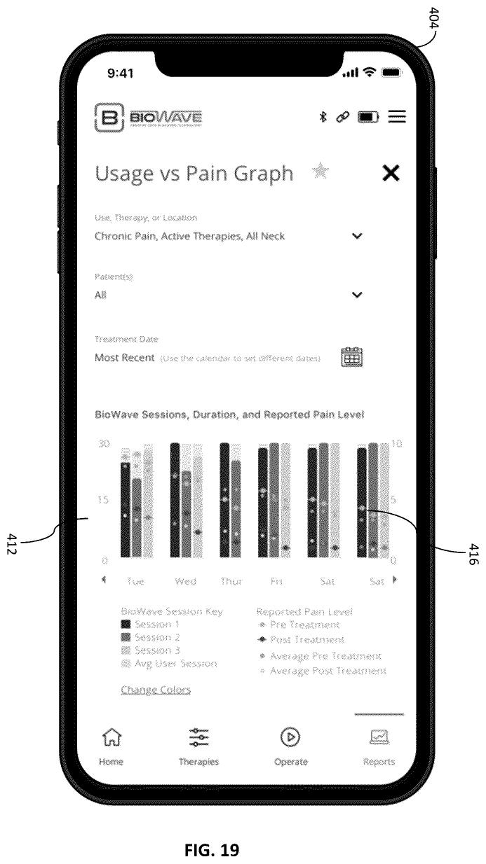

The patient facing application 412 may also be configured to ask questions of the usage regarding a pre-therapy session pain score, a post-therapy session pain score, other concomitant treatments, average duration of pain relief after a therapy session, an activities of daily living (ADL) score, the effect on pain medication consumption, any quality of life improvement, mood improvement, and activity duration such as if the user is sleeping or sitting longer or if the user is walking further than before use of the electrotherapy device 402. This requested patient information 416 can then be used in visuals and/or graphs showing the user, clinician, or others (e.g., insurance companies, hospitals, health care systems, etc.) the effectiveness of the electrotherapy device 402. The requested patient information 416 above may also be used to build visuals showing the effectiveness of the electrotherapy device 402, such as the usage vs. pain graph illustrated in FIG. 19 and percentage of therapy plan adherence just to give a few examples. The patient facing application 412 may also be configured to provide a social comparison to a user to show the adherence and effectiveness of a therapy plan that has been reported by other users or patients with the same or similar therapy plans.

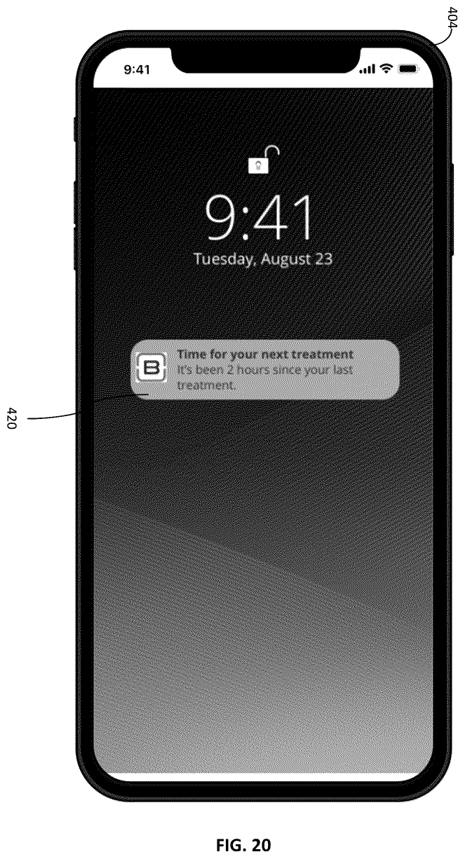

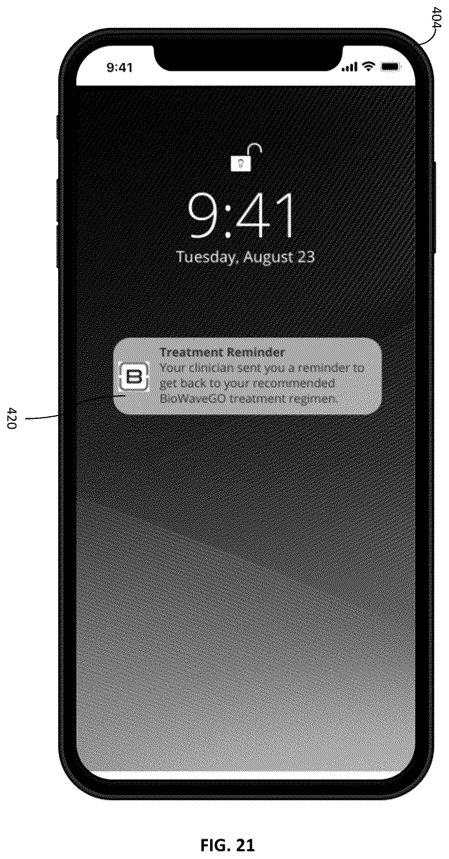

Referring now to FIGS. 20-21, which illustrate a patient computing device 404 with a plurality of notifications from the patient facing application 412 in accordance with some embodiments. In some embodiments, the patient facing application 412 may be configured to send user notifications 420 on the patient computing device 404. Such user notifications 420 may include reminders to start a therapy session as illustrated in FIGS. 20-21. Other user notifications 420 may include electrotherapy device 402 usage directions/tips, therapy recommendations 418, cautions/warnings, clinician therapy plan changes, and clinician compliancy notifications just to give a few examples. One of ordinary skill in the art will appreciate that other user notifications 420 may be displayed to a user of a patient computing device 404.

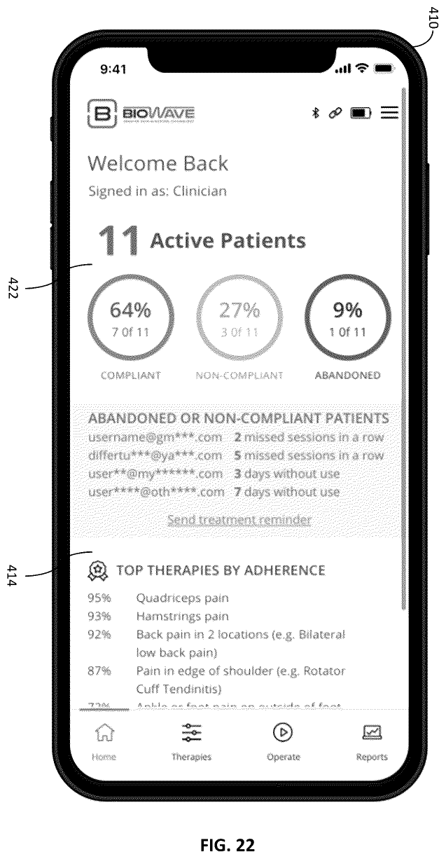

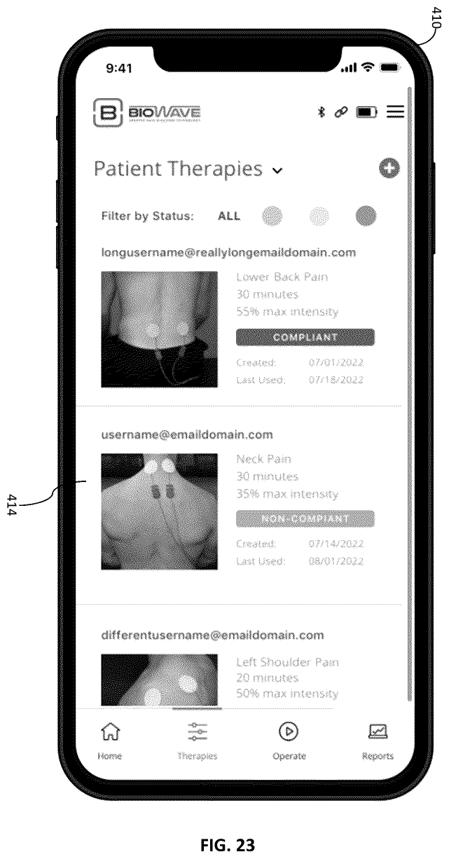

Referring now to FIGS. 22-25, which show an exemplary clinician computing device 410 with a clinician facing application 414 in accordance with some embodiments. A clinician can add patients to their clinician account by emailing an invitation to their patient from within the clinician facing application 414. The patient can accept the email invitation, which will give the clinician facing application 414 permission to download all past and future patient information 416 into the clinician's account for the clinician to track the progress of that patient on the clinician facing application 414. Once the clinician has added the patient to the clinician's account, the clinician computing device 410 may retrieve patient information 416 through wired and/or network communication with the patient information database 408 and/or patient computing device 404 through the clinician facing application 414. For example, the clinician facing application 414 may display the number of active patients on a therapy plan for using an electrotherapy device 402 on a dashboard 422. The dashboard 422 may be configured to display the number and/or percentage of patients that are compliant, non-compliant, or have abandoned their therapy plan altogether as illustrated in FIG. 22. The clinician may also be able to further drill down to see each individual therapy plan prescribed to each user under their care on their clinician facing application 414 as illustrated in FIG. 23. The clinician may then be given the option send a compliancy notification to the non-compliant patients and/or those that have abandoned their prescribed therapy plan through the clinician facing application, such as user notification 420. It is also contemplated that that the clinician may also have the option to send compliant patients user notifications 420 congratulating them for maintaining compliance with their therapy plan.

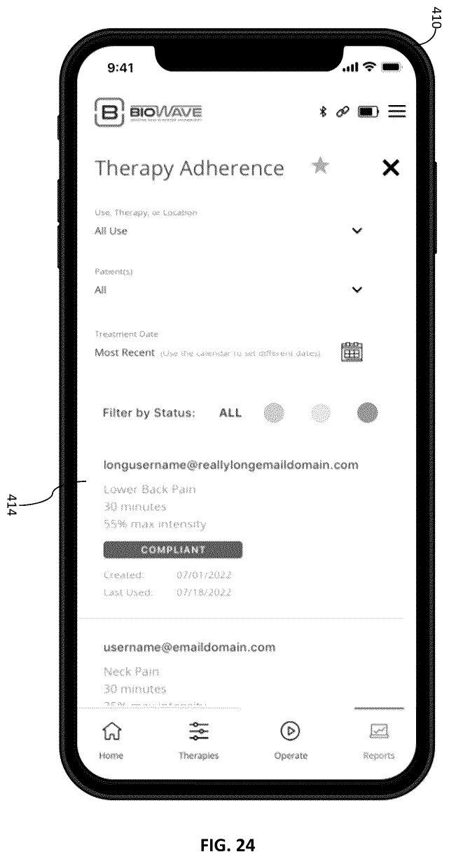

The clinician facing application 414 may also be configured to display the top therapies by adherence to the prescribed therapy plan to give the clinician feedback on what therapy plans may not be working or need to be adjusted. The clinician may also use the clinician facing application 414 to drill down and see adherence to a prescribed therapy plan for each body part and/or patient over a pre-determined period of time, such as over a day, week, month, year, etc., as illustrated in FIG. 24. In order to maximize the effectiveness of the electrotherapy device 402 and maintain a high level of compliancy with prescribed therapy plans, the clinician facing application 414 may be configured to see all of the active and passive patient information 416 described above. For example, the clinician may be able to see the visuals discussed above, such as specific patient reported pain scores pre-therapy session and post-therapy session and/or the usage vs. pain graph over a plurality of therapy sessions just to give a few examples.

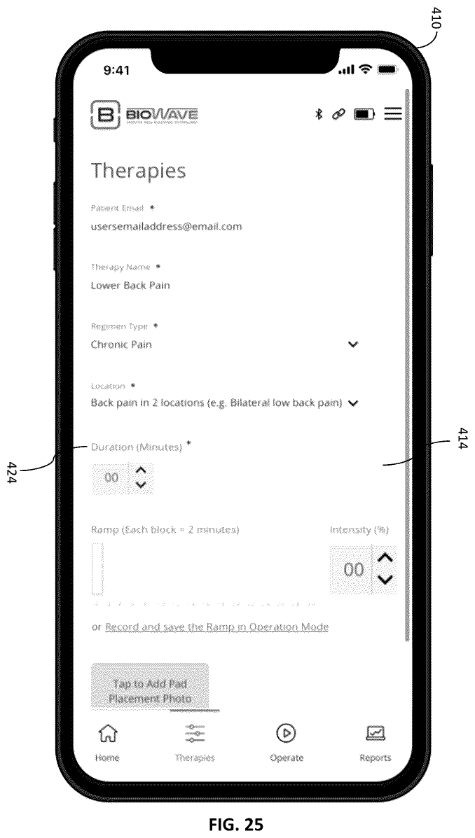

In some embodiments, the clinician may be able to provide new therapy plans to their patients through the clinician facing application 414 by creating, editing, and deleting therapy plans and/or specific therapy sessions based on the specific needs of each individual patient as illustrated in FIG. 25. For example, the clinician may determine based on the passive and active patient information 416 that a change to a specific patient's therapy plan needs to be altered. The clinician may see on their clinician facing application 414 that patient A is reporting high pain scores pre-therapy session and post-therapy session. The clinician can then create a new therapy plan, edit the current therapy plan, and/or delete old therapy plans to help maximize the use of the electrotherapy device 402 and improve patient A's outcome. For example, the clinician therapy plan change 424 may include a change to a therapy session parameter similar to the therapy recommendations 418 from the patient information database 408. Such therapy session parameters may include duration of the therapy session, ramp up/down in intensity, maximum intensity, average intensity, and/or frequency of therapy sessions just to give a few examples. After the clinician makes a change to the therapy plan for the patient, the clinician therapy plan change 424 can be transmitted to the patient's patient facing application 412 either directly to the patient facing application 412 through network 406 or stored on the patient information database 408 to later be retrieved by the patient facing application 412 through network 406.

It is also contemplated that the clinician facing application 414 may also be able to operate an electrotherapy device 402, similar to that described above regarding the patient facing application 412. The clinician computing device 410 may be configured to connect to an electrotherapy device 402 either through wired or wireless (e.g., Wi-Fi or Bluetooth®) connection in order to operate the electrotherapy device 402. For example, it may be necessary for the clinician to connect to an electrotherapy device 402 in order to demonstrate how to use the electrotherapy device 402 to a new patient.

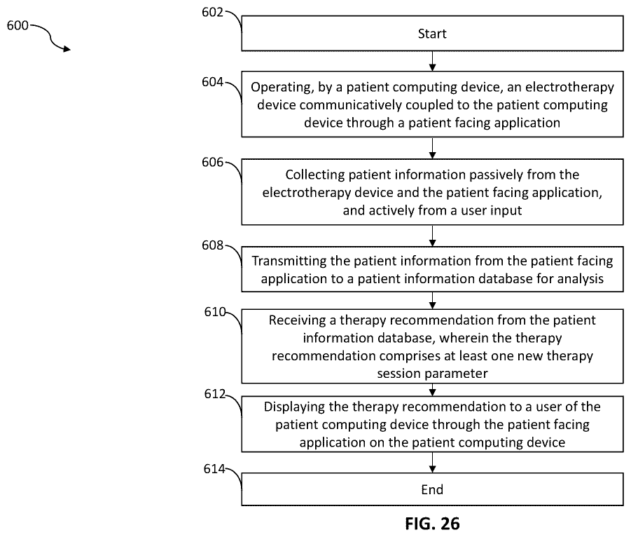

FIG. 26 is a flow chart depicting an example implementation of a set of instructions 600 to provide a therapy recommendation 418 in accordance with some embodiments. The set of instructions 600 are stored on a non-transitory computer readable medium, such as instruction memory 510 and/or working memory 512, and cause the computing device 500 to perform operations corresponding to the set of instructions 600. The set of instructions 600 may start with step 602 and moves to step 604, where the computing device 500, such as patient computing device 404, performs the operation of operating, by a patient computing device 404, an electrotherapy device 402 communicatively coupled to the patient computing device 404 through a patient facing application 412.

Continuing to refer to the set of instructions 600, at step 606 the patient computing device 404 may perform the operation of collecting patient information 416 passively from the electrotherapy device 402 and the patient facing application 412, and actively from a user input. At step 608, the patient computing device 404 may perform the operation of transmitting the patient information 416 from the patient facing application 412 to a patient information database 408 for analysis. In some embodiments, the analysis by the patient information database 408 may be through the use of algorithms, lookup tables, machine learned models, and/or artificial intelligence models. At step 610, the patient computing device 404 may perform the operation of receiving a therapy recommendation 418 from the patient information database 408, wherein the therapy recommendation 418 comprises at least one new therapy session parameter. The therapy session parameter changes from the therapy recommendation 418 may include a change in duration of a therapy session, a change in intensity (maximum, average, and/or ramp up/down in intensity), and the number of therapy sessions in a given day or week just to give a few examples. At step 612, the patient computing device 404 may perform the operation the operation of displaying the therapy recommendation 418 to a user of the patient computing device 404 through the patient facing application 412 on the patient computing device 404. The operation of the patient computing device 404 then ends at step 614.

In accordance with some embodiments, a pain management system may include a electrotherapy device, wherein the electrotherapy device is configured to generate a first signal and a second signal. Each signal may have a base frequency value between 100 Hz and 500 kHz and are amplified by a respective amplifier. The electrotherapy device may also be configured to minimize the direct current (DC) component of the first signal and the second signal using a balanced amplifier. The electrotherapy device may also be configured to form a therapeutic signal configured to reduce pain at a treatment site by simultaneously sending the first signal from a first electrode to a second electrode and sending the second signal from the second electrode to the first electrode, and then simultaneously sending the first signal from the second electrode back to the first electrode and the second signal from the first electrode back to the second electrode. The first signal and the second signal may be linearly independent off phase alternating current signals. The electrotherapy device may also be configured to adjust the therapeutic signal utilizing a feedback system based on impedance changes within a body of a patient, wherein the impedance is measured across the first electrode and the second electrode. The pain management system may also include a patient computing device communicatively coupled to the electrotherapy device through a patient facing application, wherein the patient facing application is configured to operate the electrotherapy device, display directions, collect patient information, and display therapy recommendations. The pain management system may also include a clinician computing device communicatively coupled to the patient computing device comprising a clinician facing application configured to receive transmitted patient information. The pain management system may also include a patient information database communicatively coupled to the patient computing device and the clinician computing device through the patient facing application and clinician facing application. The patient information database may be configured to receive transmitted patient information. The patient information database may be configured to store and analyze the transmitted patient information, and provides at least one therapy recommendation to a user of the patient computing device through the patient facing application based on the analysis of the transmitted patient information.

In accordance with some embodiments, the first signal and the second signal may be summed before being amplified.

In accordance with some embodiments, the respective amplifiers may be class D switching amplifiers.

In accordance with some embodiments, the therapeutic signal may be a linear combination of the first signal and the second signal.

In accordance with some embodiments, the first signal and the second signal may have a frequency difference between 1 Hz and 400 Hz.

In accordance with some embodiments, the feedback system may be configured to monitor, across the first electrode and the second electrode, at least one of a voltage or a current associated with the impedance of the body of the patient and control the therapeutic signal in response to a change in the impedance.

In accordance with some embodiments, the feedback system may utilize software configured to determine whether a change in the therapeutic signal is required, based at least in part on at least one of the voltage and the current.

In accordance with some embodiments, the base frequency value of the first signal and the second signal may be between 200 Hz and 1 kHz.

In accordance with some embodiments, the feedback system may include a resistor or current transformer that monitors a current through the body of the user. The feedback system may also include an amplifier for differentially detecting a voltage developed by the current passing through the resistor. The feedback system may also include a gain block for further amplifying the detected voltage. The feedback system may also include a buffered attenuator for sampling the voltage across the first electrode and the second electrode and setting a value of the voltage to within a predetermined range. The feedback system may also include an analog multiplexer having as a first input thereto an output of the gain block and having as a second input thereto an output of the buffered attenuator, the analog multiplexer configured to selectively output either the first input or the second input, based on a signal from a CPU. The feedback system may also include a root mean square (RMS) to DC converter having input thereto for an output of the analog multiplexer, and being configured to output a DC level approximately equal to an RMS value of the therapeutic signal. The feedback system may also include an analog-to-digital converter (ADC) configured to convert an analog output of the RMS to DC converter into a digital signal for use by a digital attenuator. The digital attenuator may also be configured to change the DC level, as required by the feedback system.

In accordance with some embodiments, the pain management system may also include a timer to monitor a treatment time set by the user when the electrotherapy device is initialized.

In accordance with some embodiments, the patient information may include patient information collected by the electrotherapy device and the patient facing application, and user input patient information entered into the patient facing application.

In accordance with some embodiments, the patient information database may be configured to provide the at least one therapy recommendation based on at least one of: an algorithm, a lookup table, a machine learned model, and an artificial intelligence model.

In accordance with some embodiments, the patient facing application may be configured to provide a previously stored treatment for use by the user.

In accordance with some embodiments, the clinician facing application may be configured to allow the clinician to send a clinician therapy plan change to the user, through the patient facing application, to provide at least one change to a therapy session parameter.

In accordance with some embodiments, the clinician facing application may be configured to receive a plurality of patient compliancy information from the patient information database.

In accordance with some embodiments, the clinician facing application may be configured to allow the clinician to send a notification message to at least one user of the patient computing device, through the patient facing application, to regain compliancy with a therapy plan.

In accordance with some embodiments, a method for managing pain of a patient may include operating, by a patient computing device, an electrotherapy device communicatively coupled to the patient computing device through a patient facing application. The method may further include collecting a plurality of patient information passively from the electrotherapy device and the patient facing application, and actively from a user input. The method may further include transmitting the plurality of patient information from the patient facing application to a patient information database for analysis. The method may further include receiving a therapy recommendation from the patient information database, wherein the therapy recommendation comprises at least one new therapy session parameter. The method may further include displaying the therapy recommendation to a user of the patient computing device through the patient facing application on the patient computing device.

In accordance with some embodiments, the method may also include sending the plurality of patient information from the patient computing device, through the patient information database, and to a clinician facing application on a clinician computing device communicatively coupled to the patient information database. The clinician facing application may be configured to display a plurality of patient compliancy data and the plurality of patient information.

In accordance with some embodiments, the method may also include receiving a clinician therapy plan change, based on the plurality of patient information and the plurality of patient compliancy data, from the clinician computing device through the patient facing application. The method may also include displaying the clinician therapy plan change to the user of the patient computing device.

In accordance with some embodiments, the method may also include notifying, through the patient facing application on the patient computing device, the user of the patient computing device to regain therapy compliance.

Although the methods described above are with reference to a flowchart, it will be appreciated that many other ways of performing the acts associated with the methods can be used. For example, the order of some operations may be changed, and some of the operations described may be optional.

In addition, the methods and system described herein can be at least partially embodied in the form of computer-implemented processes and apparatus for practicing those processes. The disclosed methods may also be at least partially embodied in the form of tangible, non-transitory machine-readable storage media encoded with computer program code. For example, the steps of the methods can be embodied in hardware, in executable instructions executed by a processor (e.g., software), or a combination of the two. The media may include, for example, RAMs, ROMs, CD-ROMs, DVD-ROMs, BD-ROMs, hard disk drives, flash memories, or any other non-transitory machine-readable storage medium. When the computer program code is loaded into and executed by a computer, the computer becomes an apparatus for practicing the method. The methods may also be at least partially embodied in the form of a computer into which computer program code is loaded or executed, such that, the computer becomes a special purpose computer for practicing the methods. When implemented on a general-purpose processor, the computer program code segments configure the processor to create specific logic circuits. The methods may alternatively be at least partially embodied in application specific integrated circuits for performing the methods.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the Wi-Fi wireless networking standard) and IEEE Standard 802.3-2015 (also known as the ETHERNET wired networking standard). Examples of a WPAN are the Bluetooth® wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term machine learned model, as used herein, includes data models created using machine learning. Machine learning, according to the present disclosure, may involve putting a model through supervised or unsupervised training. Machine learning can include models that may be trained to learn relationships between various groups of data. Machine learned models may be based on a set of algorithms that are designed to model abstractions in data by using a number of processing layers. The processing layers may be made up of non-linear transformations. The models may include, for example, artificial intelligence, neural networks, deep convolutional and recurrent neural networks. Such neural networks may be made of up of levels of trainable filters, transformations, projections, hashing, pooling and regularization. The models may be used in large-scale relationship-recognition tasks. The models can be created by using various open-source and proprietary machine learning tools known to those of ordinary skill in the art.

The foregoing is provided for purposes of illustrating, explaining, and describing embodiments of these disclosures. Modifications and adaptations to these embodiments will be apparent to those skilled in the art and may be made without departing from the scope or spirit of these disclosures.

It may be emphasized that the above-described embodiments, are merely possible examples of implementations, and merely set forth a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and the present disclosure and protected by the following claims.

Embodiments of the subject matter and the functional operations described in this specification may be implemented in electrical or electromechanical means, including the structures disclosed in this specification and their structural equivalents, or in combinations of one or more of them. Embodiments of the subject matter described in this specification may be implemented as an electrical or electromechanical unit.

It may be emphasized that the above-described embodiments, particularly any “preferred” embodiments, are merely possible examples of implementations and merely set forth for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

While this specification contains many specifics, these should not be construed as limitations on the scope of any disclosures, but rather as descriptions of features that may be specific to a particular embodiment. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

While various embodiments have been described, it is to be understood that the embodiments described are illustrative only and that the scope of the subject matter is to be accorded a full range of equivalents, many variations and modifications naturally occurring to those of skill in the art from a perusal hereof.

What is claimed is:

1. A pain management system comprising:
an electrotherapy device, wherein the electrotherapy device is configured to:
generate a first signal and a second signal, wherein each signal has a base frequency value of 100 Hz-500 kHz and are amplified by a respective amplifier;
minimize a direct current (DC) component of the first signal and the second signal using a balanced amplifier;
form a therapeutic signal configured to reduce pain at a treatment site by simultaneously sending the first signal from a first electrode to a second electrode and sending the second signal from the second electrode to the first electrode, and then simultaneously sending the first signal from the second electrode back to the first electrode and the second signal from the first electrode back to the second electrode, wherein the first signal and the second signal are linearly independent off phase alternating current signals;
adjust the therapeutic signal utilizing a feedback system based on impedance changes within a body of a patient, wherein the impedance is measured across the first electrode and the second electrode;
a patient computing device communicatively coupled to the electrotherapy device through a patient facing application, wherein the patient facing application is configured to operate the electrotherapy device, display directions, collect patient information, and display therapy recommendations;
a clinician computing device communicatively coupled to the patient computing device comprising a clinician facing application configured to receive transmitted patient information; and
a patient information database communicatively coupled to the patient computing device and the clinician computing device through the patient facing application and clinician application, wherein the patient information database is configured to receive transmitted patient information, and wherein the patient information database is configured to store and analyze the transmitted patient information, and provides at least one therapy recommendation to a user of the patient computing device through the patient facing application based on the analysis of the transmitted patient information.

2. The pain management system of claim 1, wherein the first signal and the second signal are summed before being amplified.

3. The pain management system of claim 1, wherein the respective amplifiers are class D switching amplifiers.

4. The pain management system of claim 1, wherein the therapeutic signal is a linear combination of the first signal and the second signal.

5. The pain management system of claim 1, wherein the first signal and the second signal have a frequency difference between 1 Hz and 400 Hz.

6. The pain management system of claim 1, wherein the feedback system is configured to monitor, across the first electrode and the second electrode, at least one of a voltage or a current associated with the impedance of the body of the user and control the therapeutic signal in response to a change in the impedance.

7. The pain management system of claim 6, wherein the feedback system utilizes software configured to determine whether a change in the therapeutic signal is required, based at least in part on at least one of the voltage and the current.

8. The pain management system of claim 1, wherein the base frequency value of the first signal and the second signal is between 200 Hz and 1 kHz.

9. The pain management system of claim 1, wherein the feedback system comprises:
a resistor or current transformer that monitors a current through the body of the user;
an amplifier for differentially detecting a voltage developed by the current passing through the resistor;
a gain block for further amplifying the detected voltage;
a buffered attenuator for sampling the voltage across the first electrode and the second electrode and setting a value of the voltage to within a predetermined range;
an analog multiplexer having as a first input thereto an output of the gain block and having as a second input thereto an output of the buffered attenuator, the analog multiplexer configured to selectively output either the first input or the second input, based on a signal from a CPU;
a root mean square (RMS) to DC converter having input thereto for an output of the analog multiplexer, and being configured to output a DC level approximately equal to an RMS value of the therapeutic signal;
an analog-to-digital converter (ADC) configured to convert an analog output of the RMS to DC converter into a digital signal for use by a digital attenuator; and
the digital attenuator configured to change the DC level, as required by the feedback system.

10. The pain management system of claim 1, further comprising a timer to monitor a treatment time set by the user when the electrotherapy device is initialized.

11. The pain management system of claim 1, wherein the patient information comprises patient information collected by the electrotherapy device and the patient facing application, and user input patient information entered into the patient facing application.

12. The pain management system of claim 1, wherein the patient information database is configured to provide the at least one therapy recommendation based on at least one of: an algorithm, a lookup table, a machine learned model, and an artificial intelligence model.

13. The pain management system of claim 1, wherein the patient facing application is configured to provide a previously stored treatment for use by the user.

14. The pain management system of claim 1, wherein the clinician facing application is configured to allow the clinician to send a clinician therapy plan change to the user, through the patient facing application, to provide at least one change to a therapy session parameter.

15. The pain management system of claim 1, wherein the clinician facing application is configured to receive a plurality of patient compliancy information from the patient information database.

16. The pain management system of claim 1, wherein the clinician facing application is configured to allow the clinician to send a notification message to at least one user of the patient computing device, through the patient facing application, to regain compliancy with a therapy plan.

17. A method for managing pain of a patient comprising:
operating, by a patient computing device, an electrotherapy device communicatively coupled to the patient computing device through a patient facing application;
collecting a plurality of patient information passively from the electrotherapy device and the patient facing application, and actively from a user input;
transmitting the plurality of patient information from the patient facing application to a patient information database for analysis;
receiving a therapy recommendation from the patient information database, wherein the therapy recommendation comprises at least one new therapy session parameter; and
displaying the therapy recommendation to a user of the patient computing device through the patient facing application on the patient computing device.

18. The method of claim 17, further comprising sending the plurality of patient information from the patient computing device, through the patient information database, and to a clinician facing application on a clinician computing device communicatively coupled to the patient information database, wherein the clinician facing application is configured to display a plurality of patient compliancy data and the plurality of patient information.

19. The method of claim 18, further comprising:
receiving a clinician therapy plan change, based on the plurality of patient information and the plurality of patient compliancy data, from the clinician computing device through the patient facing application; and
displaying the clinician therapy plan change to the user of the patient computing device.

20. The method of claim 18, further comprising notifying, through the patient facing application on the patient computing device, the user of the patient computing device to regain therapy compliance.
